(19) United States
(12) Patent Application Publication
(10) Pub. No.: US 2015/0285817 A1
(43) Pub. Date: Oct. 8, 2015

(54) Method for treating and identifying lung cancer patients likely to benefit from EGFR inhibitor and a monoclonal antibody HGF inhibitor combination therapy

(71) Applicants: Biodesix, Inc., Boulder, CO (US); AVEO Pharmaceuticals, Inc., Cambridge, MA (US)

(72) Inventors: Heinrich Röder, Steamboat Springs, CO (US); Julia Grigorieva, Steamboat Springs, CO (US); May Han, Brookline, MA (US); Philip Komarnitsky, Chestnut Hill, MA (US); Jeno Gyuris, Lincoln, MA (US)

(21) Appl. No.: 14/678,428

(22) Filed: Apr. 3, 2015

Related U.S. Application Data
(60) Provisional application No. 61/976,844, filed on Apr. 8, 2014.
(60) Provisional application No. 61/976,849, filed on Apr. 8, 2014.
(60) Provisional application No. 62/080,611, filed on Nov. 17, 2014.
(60) Provisional application No. 62/080,616, filed on Nov. 17, 2014.

(51) Int. Cl. G01N 33/68; G06F 19/00; A61K 31/517; A61K 39/395; A61K 31/5377

(52) U.S. Cl. CPC G01N 33/6848; A61K 39/3955; A61K 31/5377; A61K 31/517; G06F 19/34; G01N 2333/46; G01N 2800/7028; A61K 2039/505

(57) ABSTRACT

A test to identify whether a lung patient is likely to benefit from combination therapy in the form of an epidermal growth factor receptor inhibitor (EGFR-I) and a monoclonal antibody drug targeting hepatocyte growth factor (HGF) as compared to EGFR-I monotherapy. The test makes use of a mass spectrum obtained from a serum or plasma sample and a computer configured as a classifier operating on the mass spectrum and a training set in the form of class-labeled mass spectra from other cancer patients. The computer classifier executes a classification algorithm, such as K-nearest neighbor, and assigns a class label to the serum or plasma sample. Samples classified as “Poor” or the equivalent are associated with patients which are likely to benefit from the combination therapy more than from EGFR-I monotherapy. The invention also includes improved methods of treating patients predicted by the test.

### PRIORITY

This application claims priority benefits pursuant to 35 U.S.C. §119 to U.S. provisional application Ser. Nos. 61/976,844 and 61/976,849, both filed on Apr. 8, 2014, and U.S. provisional application Ser. Nos. 62/080,611 and 62/080,616, both filed Nov. 17, 2014.

### BACKGROUND

This invention relates to the fields of biomarker discovery and personalized medicine, and more particularly relates to a method for predicting, in advance of treatment, whether a non-small-cell lung cancer (NSCLS) patient is likely to obtain benefit from combination treatment in the form of an epidermal growth factor receptor inhibitor (EGFR-I) such as gefitinib in combination with a monoclonal antibody drug targeting hepatocyte growth factor (HGF), such as for example ficlatuzumab, as compared to treatment by an EGFR-I alone. Ficlatuzumab is a humanized HGF inhibitory monoclonal antibody which binds to HGF, the only known ligand for the c-Met receptor.

Non-Small-Cell Lung Cancer is a leading cause of death from cancer in both men and women in the United States. There are at least four (4) distinct types of NSCLC, including adenocarcinoma, squamous cell, large cell, and bronchioalveolar carcinoma. Adenocarcinoma of the lung accounts for over 50% of all lung cancer cases in the U.S. This cancer is more common in women and is still the most frequent type seen in non-smokers. Squamous cell (epidermoid) carcinoma of the lung is a microscopic type of cancer most frequently related to smoking Large cell carcinoma, especially those with neuroendocrine features, is commonly associated with spread of tumors to the brain. When NSCLC tumor cells enter the blood stream, cancer can spread to distant sites such as the liver, bones, brain, and other places in the lung.

Treatment of NSCLC can take several forms. While surgery is the most potentially curative therapeutic option for NSCLC, it is possible only in early stages. Chemotherapy is the mainstay treatment of advanced cancers.

Recent approaches for developing anti-cancer drugs to treat the NSCLC patient focus on reducing or eliminating the ability for cancer cells to grow and divide. These anti-cancer drugs are used to disrupt the signals to the cells which tell them to grow. Normally, cell growth is tightly controlled by the signals that the cells receive. In cancer, however, this signaling goes wrong and the cells continue to grow and divide in an uncontrollable fashion, thereby forming a tumor. One of these signaling pathways begins when a chemical in the body, called epidermal growth factor, binds to a receptor that is found on the surface of many cells in the body. The receptor, known as the epidermal growth factor receptor (EGFR) sends signals to the cells, through the activation of tyrosine kinase (TK), a cytoplasmic domain in EGFR, which is found within the cells. The signals are used to notify cells to grow and divide.

Two anti-cancer drugs that were developed and prescribed to the NSCLC patients are called gefitinib (trade name “Iressa”®, AstraZeneca, London UK) and erlotinib (trade name “Tarceva”®, OSI Pharmaceuticals, Farmingdale N.Y.). These anti-cancer drugs target the EGFR pathway and have shown promise in being effective towards treating NSCLC cancer. Iressa inhibits tyrosine kinase that is present in lung cancer cells, as well as other cancers and normal tissues, and that appears to be especially important to the growth of cancer cells. Iressa and Tarceva have been used as a single agent monotherapy for treatment of NSCLC that has progressed after, or failed to respond to, two other types of chemotherapies and in the front-line treatment of patients whose tumors exhibit mutations in the EGFR.

Biodesix Inc., Boulder Colo., has developed a test known as VeriStrat® which predicts whether NSCLC patients are likely or not likely to benefit from treatment of EGFR pathway targeting drugs, including gefitinib and erlotinib. The test is described in U.S. Pat. No. 7,736,905, the content of which is incorporated by reference herein. The test is also described in Taguchi F. et al., J. Nat. Cancer Institute, 2007 v. 99 (11), 838-846, the content of which is also incorporated by reference herein. Additional applications of the test are described in other patents of Biodesix, Inc., including U.S. Pat. Nos. 7,858,380; 7,858,389 and 7,867,774, the contents of which are incorporated by reference herein.

In brief, the VeriStrat test is based on serum and/or plasma samples of cancer patients. Through a combination of MALDI-TOF mass spectrometry and data analysis algorithms implemented in a computer, it compares a set of eight features at predefined m/z ranges with those from a training cohort (“training set”) with the aid of a classification algorithm, such as the K-nearest neighbor algorithm. The classification algorithm generates a classification label for the patient sample: either VeriStrat “good”, VeriStrat “poor”, or VeriStrat “indeterminate.” In multiple clinical validation studies it has been shown that patients, whose pre-treatment serum/plasma was classified VeriStrat “good”, have significantly better outcome when treated with EGFR inhibitor drugs than those patients whose sample results in a VeriStrat “poor” classification. In few cases (less than 2%) no determination can be made, resulting in a VeriStrat “indeterminate” label. VeriStrat is commercially available from Biodesix, Inc. and is used in treatment selection for NSCLC patients in the second line setting and for frontline patients not eligible for chemotherapy.

In pending U.S. patent application publication 2011/0208433, assigned to Biodesix, Inc., incorporated by reference herein, we summarized a collection of experimental data involving the VeriStrat test across a number of different patient populations and cancer tumor types. Among other things, the application explains that the VeriStrat test shows a separation, indicating differential outcomes, with a Hazard ratio between VeriStrat good and poor subgroups of around 0.45 for EGFR inhibitor (EGFR-I) mono-therapies. This was independent of the mechanism of action of the EGFR-I, e.g. for small molecule TKIs (e.g., erlotinib, gefitinib) and antibody (receptor) inhibitor based EGFR-Is (e.g. cetuximab), independent of tumor histology, e.g. adenocarcinoma, and squamous cell carcinoma, and independent of tumor site, e.g. NSCLC, squamous cell carcinoma of the head and neck (SCCHN), and colorectal cancer (CRC). No significant correlation with other population characteristics was observed: i.e., not with genomic marker, e.g. EGFR mutation status or KRAS status, and not with certain clinical factors such as race. This application explains that VeriStrat has a strong prognostic component exhibited by a differential outcome between VeriStrat poor and VeriStrat good subgroups in the absence of treatment.

All this leads to the conclusion that VeriStrat poor classification defines a novel disease state of clinical significance (worse prognosis) in solid epithelial tumors. The observed phenomena allowed for some tentative conclusions on the molecular state of VeriStrat poor tumors: As EGFR-Is are not effective, as the effect is the same for both TKIs and antibody based therapies, it is likely that in VeriStrat poor patients a pathway below the receptors and the TKI domains is different from VeriStrat good patients, i.e. upregulated. As we observed no correlation with KRAS mutation status, we further concluded that the affected pathway is below, i.e., downstream of RAS.

Most modern biomarker-based tests are very specific with respect to tumor type and histology, specific interventions, and clinico-pathological factors. For example, genetic tests based on tumor tissue like mutations in the EGFR domain, KRAS mutations, and gene copy number analysis via Fluorescence In-Situ Hybridization (FISH) appear to work only in very specific indications. While EGFR mutations are strongly correlated with objective response and progression free survival on EGFR-Is in first line NSCLC cancer with adenocarcinoma, they do not exhibit similar utility for squamous cell carcinoma due to less frequent EGFR mutations in this type of NSCLC. KRAS mutations can be associated with absence of benefit from cetuximab in colorectal cancer, but attempts to transfer this to NSCLC have been unsuccessful. There are no known validated markers for EGFR-I benefit in squamous cell cancer of the head and neck (SCCHN). The limitations of genetic tests may be related to their focus on very specific mutations that are only a small part of the complex mechanism of carcinogenesis. Also, it is further believed that these tests are based on a reductionist point of view, i.e., reducing tumor biology to just tumor cells, and ignoring the important interplay between tumor cells, the tumor supporting environment, the vascular support system, and the role of chronic inflammatory mechanisms in the micro-tumor environment.

Recently, Aveo Pharmaceuticals, Inc., Cambridge Mass., conducted a Phase II clinical trial to assess whether ficlatuzumab (also known as AV-299) in combination with gefitinib may be effective in treatment of NSCLC as compared to administration of gefitinib alone. As explained in the review article of D'Arcangelo et al., Focus on the potential role of ficlatuzumab in the treatment of non-small cell lung cancer, Biologics: Targets and Therapies 2013:7 p. 61-68, the c-Met oncogene encodes a receptor (Met, sometimes referred to as c-MET) which is a member of the tyrosine kinase family. Its only known ligand is HGF. HGF is a platelet-derived mitogen for hepatocytes and other normal cell types and a fibroblast-derived factor for epithelial cell scattering, i.e., induces random movement of epithelial cells. HGF is a morphogen that induces transition of epithelial cells into a mesenchymal morphology. c-Met/HGF pathway activation has been implicated in EGFR-TKI resistance in lung adenocarcinoma. Ficlatuzumab is an HGF inhibitory monoclonal antibody (mAb) that prevents c-Met receptor activation by blocking its ligand, HGF. See FIG. 1. See U.S. Pat. Nos. 8,580,930; 8,273,355; 7,943,344; and 7,649,083, which describe exemplary humanized anti-HGF antibodies, including humanized forms of the murine 2B8 monoclonal, namely HE2B8-1, HE2B-2, HE2B8-3 and HE2B8-4 (Ficlatuzumab), among others.

In a presentation at the 2012 European Society of Medical Oncology Annual Meeting (Sep. 28-Oct. 2, 2012), Vienna Austria, Dr. Tony Mok et al. presented a poster paper describing their finding from the Phase II study of ficlatuzumab in combination with gefitinib versus gefitinib alone in treatment of NSCLC. In the intent to treat population the trial did not show a significant advantage of the combination therapy over monotherapy treatment. The above-listed patents and poster paper are incorporated by reference herein. The investigators explored a number of different biomarkers using immunohistochemical and PCR methods and found, among other things, that the addition of ficlatuzumab to gefitinib may prolong overall survival in patients with high stromal HGF expression, although it should be noted that the addition of ficlatuzumab to gefitinib did not appear to prolong progression-free survival in this patient subset. Furthermore, less than 70% of patients with tissue samples were able to be tested for stromal HGF expression, partially due to the challenging nature of the assay, including the availability of stromal tissue in the tumor samples collected.

Given the limitations described above, it would be desirable to (1) have a more accurate predictor of efficacy and (2) be able to rapidly and reliably identify, in advance of treatment, a patient as being likely to benefit from combination therapy in the form of a monoclonal antibody drug targeting HGF and an EGFR-I as compared to EGFR-I monotherapy, without having to measure directly stromal HGF or other tumor derived biomarker level, or biomarkers based on immunohistochemical testing methods. This invention meets that need.

In a previously filed U.S. patent application publication 2011/0208433, discussed above, it was postulated that the VeriStrat test could be used to identify patients that may benefit from MET inhibitors, such as, for example, AV-299 (ficlatuzumab) but the document does not identify a method for selection of patients likely to obtain benefit from EGFR-I and anti-HGF combination therapy as compared to EGFR-I monotherapy,

### SUMMARY

The present invention can be understood as an improvement or enhancement of the VeriStrat test of the applicants' assignee, in that we have found from the VeriStrat test a combination therapy that benefits those NSCLC patients whose blood samples are classified as “poor” or the equivalent. In particular, in a first aspect, a method is disclosed for predicting whether a NSCLC patient is a member of a class of cancer patients likely to benefit from a treatment for NSCLC in the form of administration of a combination therapy in the form of an epidermal growth factor receptor inhibitor (EGFR-I) and a monoclonal antibody drug targeting HGF as compared to EGFR-I monotherapy. The method makes use of a serum or plasma sample, mass spectrometry and a programmed computer. The method, which can be considered to be a predictive test, can be conducted rapidly from a simple blood sample.

The method includes the steps of:

(a) storing in a computer readable medium a reference set comprising data in the form of class-labeled mass spectra obtained from a multitude of cancer patients, the class-labels of the form GOOD or the equivalent indicating the patient had stable disease six months after initiating treatment of the cancer with an EGFR-I and POOR or the equivalent indicating the patients had early progression of disease after initiating treatment of the cancer with an EGFR-I; (Note, in this document use the expression “or the equivalent” to signify that the particular class label moniker that is used is not important, for example “Benefit”, “+” and so forth would be considered equivalent to a “Good” class label, and “Non-benefit”, “−” and so forth would be considered equivalent to a Poor class label. Any convenient binary classification label regime is possible and considered equivalent to GOOD and POOR.)

(b) providing a serum or plasma sample from the NSCLC patient to a mass spectrometer and conducting mass spectrometry on the serum or plasma sample and thereby generating a mass spectrum for the serum or plasma sample;

(c) conducting pre-defined pre-processing steps on the mass spectrum obtained in step b) with the aid of a programmed computer;

(d) obtaining integrated intensity values of selected features in the mass spectrum at one or more predefined m/z ranges after the pre-processing steps on the mass spectrum recited in step c) have been performed; and

(e) executing in the programmed computer a classification algorithm operating on both the integrated intensity values obtained in step (d) and the reference data set stored in step (a) and responsively generating a class label for the serum or plasma sample.

Surprisingly, we have discovered that if the class label generated in step (e) is POOR or the equivalent, the patient is identified as being likely to benefit from the combination treatment. In this respect, the test is an improvement to the VeriStrat test described in the Biodesix, Inc. prior U.S. Pat. No. 7,736,905, in that while the '905 patent describes the POOR class label as indicating that a patient is not likely to benefit from EGFR inhibitors in treatment of NSCLC, the POOR class label in this invention describes a class of patients that are likely to benefit from the combination of an epidermal growth factor receptor inhibitor (EGFR-I) and a monoclonal antibody drug targeting HGF, such as example the combination of gefitinib and ficlatuzumab, as compared to EGFR-I monotherapy.

The step (a) of storing the reference set is should be performed prior to the performance of steps b), c), d) and e). For example, a reference set can be defined from a set of samples subject to mass spectroscopy, using the peak finding and other methods of the U.S. Pat. No. 7,736,905, and subject to suitable validation studies, and then stored in a computer system, portable computer medium, cloud storage or other form for later use. At the time when a given serum or plasma sample is to be tested and processed in accordance with steps b)-e) the reference set is accessed and used for classification in accordance with step e).

In one particular embodiment, the EGFR-I in the combination treatment is a small molecule EGFR inhibitor such as gefitinib or other small molecule drugs targeting the EGFR pathway, e.g., erlotinib. The monoclonal antibody drug targeting HGF may take the form of a monoclonal antibody designed to bind to HGF, such as, for example, ficlatuzumab. In another embodiment, the reference set is in the form of class-labeled mass spectra obtained from a multitude of NSCLC patients. However, the class-labeled spectra could be obtained from other types of solid epithelial tumor cancer patients, such as for example, colorectal cancer patients or SCCHN cancer patients. A NSCLC reference set was used in the present example because the existing VeriStrat test already uses the NSCLC reference set, it is well characterized and was subject to extensive validation studies.

In another embodiment, the classification algorithm is in the form of a k-nearest neighbor classification algorithm. However, other classification algorithms could be used, for example margin-based classifiers, and probabilistic classifiers, and logistical combination of mini-classifiers, i.e., so-called CMC/D classifiers (Combination of Mini-Classifiers with Dropout regularization) described throughout the detailed description and figures in the pending U.S. patent application of H. Röder et al., Ser. No. 14/486,442 filed Sep. 15, 2015, which is incorporated by reference herein. In one embodiment, the predefined m/z ranges which are used for classification of the serum or plasma sample takes the form of one or more m/z ranges listed in TABLE 3, such as for example eight of the m/z ranges. It will be appreciated that other m/z ranges could be used for classification. For example, other discriminating peaks/features could be defined by subjecting a group of samples to the “deep-MALDI” mass spectrometry methods described in U.S. patent application of H. Röder et al., publication no. 2013/0320203, incorporated by reference, either alone or in conjunction with the classifier development methods of application Ser. No. 14/486,442.

In other embodiments, the present invention relates to improved methods of treating a subject with Non-Small Cell Lung Cancer (NSCLC). The improved methods comprise:

(a) predicting whether said subject with NSCLC is a member of a class of cancer patients likely to benefit from a treatment for NSCLC in the form of administration of a combination of an EGFR-I and a monoclonal antibody drug targeting hepatocyte growth factor (HGF) as compared to EGFR-I monotherapy using the method of claim 1; and

(b) if the subject is identified as being likely to benefit from the combination treatment, as compared to monotherapy, treating the subject with a combination of an EGFR-I and a monoclonal antibody drug targeting HGF.

In certain embodiments, the improved method of treatment comprises treating the subject with the combination of an EGFR-I selected from the group consisting of gefitinib, erlotinib, dacomitinib, lapatinib, afatinib, and cetuximab and a monoclonal antibody drug targeting HGF. In particular embodiments, the drug targeting the HGF is ficlatuzumab.

The skilled clinician will be able to determine the appropriate dosage amount and number of doses of agents to be administered to a subject, dependent upon both the age and weight of the subject, the underlying condition, and the response of an individual subject to the treatment. In addition, the clinician will be able to determine the appropriate timing and routes for delivery of the agent in a manner effective to treat the subject. Dosing may be done consistent with FDA-approved labeling or in accordance with clinical experience. An exemplary dose for gefitinib is a 250 mg tablet as a daily dose. Exemplary doses for erlotinib are a 25 mg, 100 mg or 150 mg tablet as a daily dose. An exemplary dosage regimen for cetuximab is 400 mg/m2 as an initial dose as a 120 minute intravenous infusion followed by 250 mg/m2 weekly, infused over 60 minutes.

A therapeutic dosage of ficlatuzumab falls within the range of from about 0.1 mg/kg to about 100 mg/kg, preferably from about 0.5 mg/kg to about 20 mg/kg. Exemplary dosage regimens for ficlatuzumab are 2 mg/kg every two weeks, 10 mg/kg, every 2 weeks, and 20 mg/kg, every 2 weeks, which is administered parenterally, e.g., by intravenous infusion.

### BRIEF DESCRIPTION OF THE DRAWINGS

FIG. 1 is an illustration of the c-Met receptor and its signaling functions, showing the monoclonal antibody ficlatuzumab binding to HFG, the ligand for the c-Met receptor.

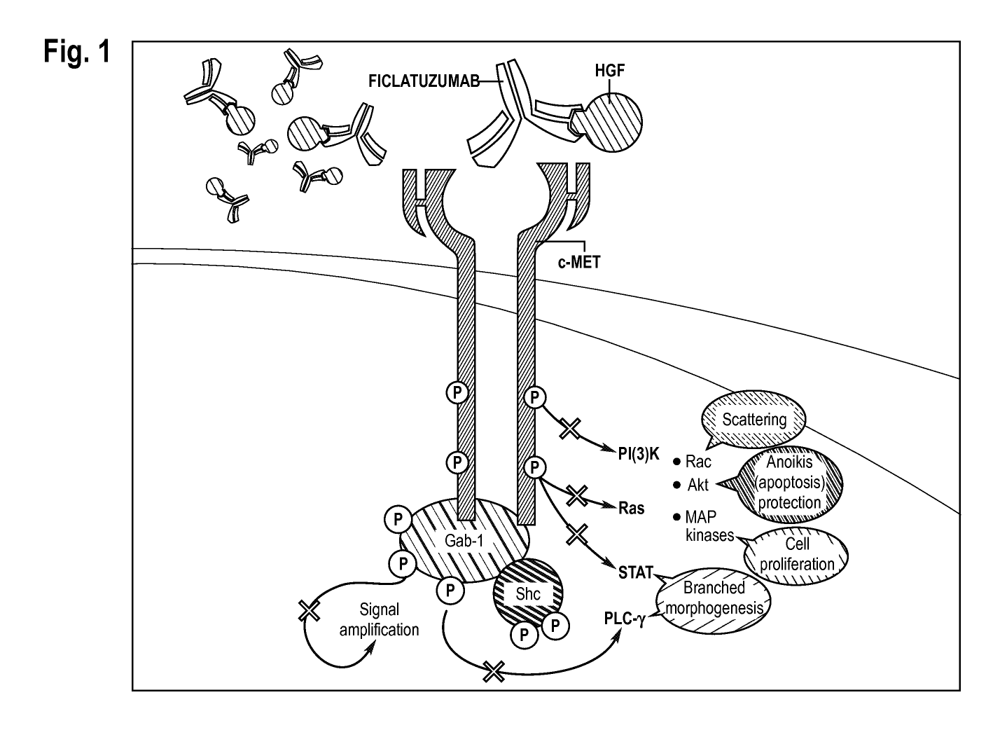

FIG. 2A is a Kaplan-Meier plot of overall survival (OS) for patients in the gefitinib arm of the Phase 2 ficlatuzumab+gefitinib study (“the Study” herein). FIG. 2B is a Kaplan-Meier plot of the progression free survival (PFS) for patients in the gefitinib arm of the Study. FIGS. 2A and 2B illustrate that the VeriStrat classification (“good”/“poor”) is prognostic for OS and PFS in the gefitinib arm, as indicated by the separation between the curves for Good and Poor patients shown in the plots of FIGS. 2A and 2B.

FIG. 3A is a plot of OS for patients in the gefitinib+ficlatuzumab arm of the Study. FIG. 3B is a plot of the PFS for patients in the gefitinib+ficlatuzumab arm of the Study. FIGS. 3A and 3B illustrate that the VeriStrat classification (“good”/“poor”) was not prognostic for OS and PFS in the gefitinib+ficlatuzumab arm, as indicated by the lack of separation between curves for the Good and Poor patients.

FIG. 4A is a plot of OS for patients in the gefitinib+ficlatuzumab arm, as compared to the gefitinib monotherapy arm, for those patients with VeriStrat poor status. FIG. 4B is a plot of the PFS for patients in the gefitinib+ficlatuzumab arm as compared for the gefitinib monotherapy arm, for those patients with VeriStrat poor status. FIGS. 4A and 4B illustrate that the patients testing VeriStrat poor in advance of treatment were likely to benefit from the addition of ficlatuzumab to gefitinib as compared to gefitinib monotherapy.

FIG. 5A is a plot of OS for patients in the gefitinib+ficlatuzumab arm, as compared to the gefitinib monotherapy arm, for those patients with VeriStrat good status. FIG. 5B is a plot of the PFS for patients in the gefitinib+ficlatuzumab arm as compared for the gefitinib monotherapy arm for VeriStrat good status patients. FIGS. 5A and 5B illustrate that the patients testing VeriStrat good in advance of treatment did not appear to benefit from the addition of ficlatuzumab to gefitinib monotherapy.

FIG. 6A is a plot of OS for patients in the gefitinib+ficlatuzumab arm, as compared to the gefitinib monotherapy arm, for those patients with VeriStrat poor status and having EGFR sensitizing mutations (EGFR SM+). FIG. 6B is a plot of the PFS for patients in the gefitinib+ficlatuzumab arm as compared for the gefitinib monotherapy arm, for those patients with VeriStrat poor, EFFR SM+ status. FIGS. 6A and 6B illustrate that the patients testing VeriStrat poor and have EGFR SM+ status were likely to benefit from the addition of ficlatuzumab to gefitinib.

FIG. 7A is a plot of OS for patients in the gefitinib arm for those patients with VeriStrat poor and VeriStrat good status, and having EGFR SM+ patients. FIG. 7B is a plot of the PFS for patients in the gefitinib arm for those patients with VeriStrat poor and VeriStrat good status, and having EFFR SM+ status.

FIG. 8A is a plot of OS for patients in the gefitinib+ficlatuzumab arm for those patients with VeriStrat poor and VeriStrat good status, and having EGFR SM+ status. FIG. 8B is a plot of the PFS for patients in the gefitinib+ficlatuzumab arm for those patients with VeriStrat poor and VeriStrat good status, and having EGFR SM+ status.

FIG. 9 is a flow chart showing the steps used in conducting a mass spectral test for predicting NSCLC patient benefit from combination treatment in the form of EGFR-I and a monoclonal antibody drug targeting HGF as compared to EGFR-I monotherapy.

### DETAILED DESCRIPTION

A test is described below which can be considered an improvement or enhancement to the VeriStrat test of Biodesix, Inc. The test is used for predicting in advance of treatment whether a NSCLC patient is a member of a class of patients that are likely to benefit from administration of a combination therapy in the form of an EGFR-I plus a monoclonal antibody drug targeting the HGF as compared to EGFR-I monotherapy. The test was developed as a result of conducting mass spectrometry testing on a set of serum or plasma samples obtained from patients enrolled in the Phase II clinical trials of ficlatazumab+gefitinib vs. gefitinib alone described in the Mok et al. poster paper cited in the background section of this document (“the Study” herein). An overview of this Study, the mass spectrometry testing we conducted, and the data demonstrating our discovery that the VeriStrat classifier is effective in identifying patients in advance of treatment that are likely to benefit (in PFS and OS) from the combination treatment as compared to EGFR-I monotherapy will be described in the sections below.

The Study

The Phase II study of ficlatazumab+gefitinib vs. gefitinib in treatment of NSCLC patents is described in the Mok et al. poster. Briefly, by way of overview, 188 patients were enrolled in the study. Key entry criteria for the study were Stage III/IV NSCLC, treatment-naïve, adenocarcinoma histology, with the patients selected from an Asian population and being either non-smokers or light former smokers. Stratification of the population was based on Eastern Cooperative Oncology Group Performance Status (ECOG PS), smoking history, and gender. 1:1 randomization of the population was performed, with one half of the patients (n=94) enrolled in a gefitinib+ficlatuzumab treatment arm (“combination arm”), the remaining half of the patients (n=94) enrolled in the gefitinib monotherapy treatment arm (“monotherapy arm”).

The treatment in the combination arm consisted of gefitinib 250 mg daily plus ficlatuzumab, 20 mg/kg, every 2 weeks in 28 day cycles. The monotherapy arm consisted of gefitinib 250 mg daily. In the monotherapy arm, crossover was permitted into the combination treatment arm in cases of patients who initially responded to gefitinib for 12 weeks or more, and subsequently exhibited disease progression. Non-responders and patients who did not consent to participate in the crossover were discontinued from the study.

The primary objective of the study was to compare the overall response rate (ORR) in Asian patients with lung adenocarcinoma receiving ficlatuzumab plus gefitinib or gefitinib alone. Key secondary objectives were to compare the response duration, progression free survival (PFS) and overall survival (OS) in patients treated alone in ITT and in biomarker-defined subgroups, including c-Met and HGF expression levels, EGFR sensitizing mutation status (EFGR SM+, SM−) and EGFR and c-Met gene copy number. Another secondary objective was to assess whether acquired resistance to gefitinib can be overcome with the addition of ficlatuzumab in patients who progressed after initially experiencing disease control in the gefitinib-alone arm.

The demographics of the patient population enrolled in the study are shown in table 1 below.

TABLE 1

| | Ficlatuzumab plus gefitinib | Gefitinib alone |
|---|---|---|
| | n = 94 | n = 94 |
| Male, n (%) | 19 (20) | 19 (20) |
| Female, n (%) | 75 (80) | 75 (80) |
| Median age, years (range) | 58 (35, 80) | 62 (25, 84) |
| Smoking, n (%) | | |
| Yes | 6 (6) | 5 (5) |
| No | 88 (94) | 89 (95) |
| ECOG PS, n (%) | | |
| 0 | 27 (29) | 26 (28) |
| 1 | 64 (68) | 65 (69) |
| 2 | 3 (3) | 3 (3) |

As noted in the Background and as reported in the Mok et al. poster paper, the investigators reported several conclusions from the Phase 2 study, including (1) addition of ficlatuzumab to gefitinib did not result in statistically significantly improved ORR or PFS in the ITT (intention-to-treat) population in Asian treatment-naïve NSCLC patients, and (2) preliminary OS results favor ficlatuzumab plus gefitinib in patients with stromal HGF high (P=0.03) and SM− (P=0.25) biomarkers.

We obtained serum or plasma samples from patients from the Study to determine whether it might be possible to identify, i.e., predict, whether a patient is likely to benefit from the combination of EGFR-I such as gefitinib in combination with a monoclonal antibody drug targeting HGF in advance of treatment from a mass-spectrometry test on a serum or plasma sample as compared to gefitinib monotherapy. We found that we were able to make such identifications. The following sections describe our research and results and explain practical implementations of a test to make such a prediction.

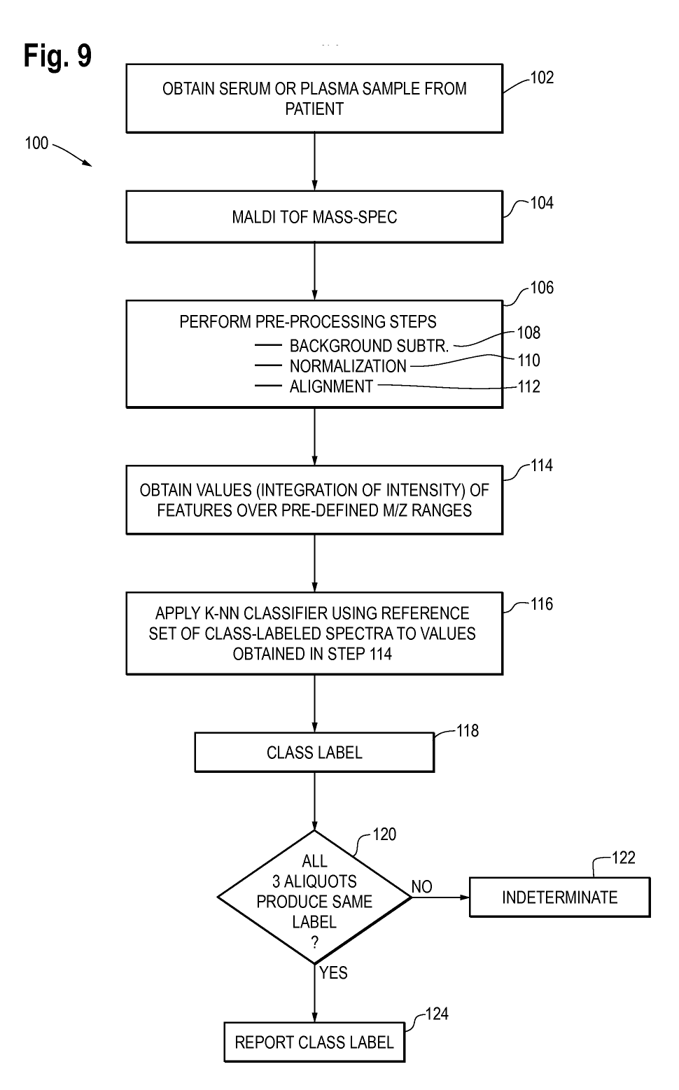

In summary, we obtained pre-treatment serum samples from all 188 patients enrolled in the study described above. The samples were blinded and subject to MALDI-TOF mass-spectrometry. The resulting mass spectra were subject to predefined pre-processing steps, described below, and integrated intensity values at pre-defined m/z positions ranges, (i.e., feature values) in the pre-processed spectra were obtained. The m/z ranges were those used in the VeriStrat test, see the explanation below and U.S. Pat. No. 7,736,905. These intensity values were supplied to a classification algorithm (k-nearest neighbor) that compared the intensity values to a reference set of class-labeled mass spectra to produce a class label for each of the samples. This process, including the classification algorithm, and reference set will be explained in further detail below in conjunction with FIG. 9. We found that those samples from the Study in which the classification algorithm produced the “poor” class label were associated with patients that were likely to benefit from the combination treatment as compared with the monotherapy arm. Those with the “good” class label were found to obtain similar benefit in both treatment cohorts.

Of the 188 patients enrolled in the study, we were able to assign VeriStrat status (good/poor) to 183 serum or plasma samples obtain pre-treatment from different patients enrolled in the study. Several samples were not available for analysis and three samples were tested “indeterminate”, i.e., the classification algorithm failed to classify three different aliquots of the sample with the same classification label, and were therefore excluded from the analysis. Key baseline characteristics of the patients for whom a class label could be assigned are shown in Table 2:

TABLE 2

| | Poor | | Good | |
|---|---|---|---|---|
| | Mono- | Combination | Mono- | Combination |
| | therapy G | F + G | therapy G | F + G |
| ECOG 0/1/2 | 1/14/2 | 2/14/2 | 25/50/1 | 22/46/1 |
| Median Age | 69 | 59 | 62 | 59 |
| Male/Female | 4/13 | 4/14 | 15/61 | 15/54 |
| Past/Non Smoker | 1/16 | 3/15 | 4/72 | 3/66 |
| EGFR NA/SM−/ | 4/7/6 | 4/9/5 | 21/23/32 | 28/13/28 |
| SM+ | | | | |

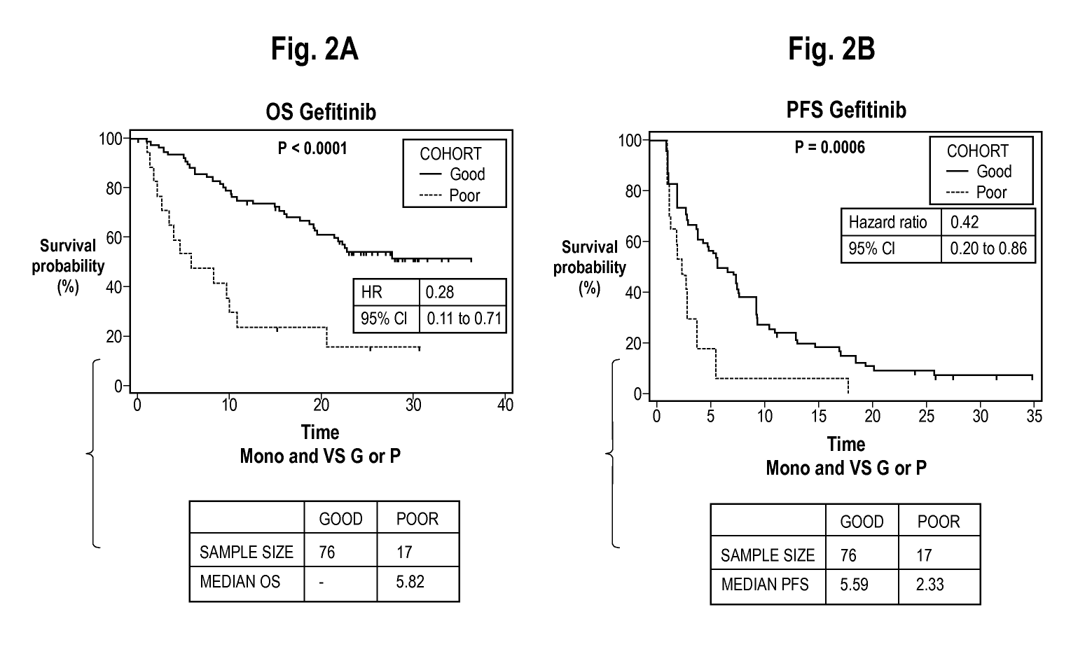

Our results showing the efficacy of the monotherapy arm in the Study, stratified by VeriStrat status (good/poor) is shown in the Kaplan-Meier plots of FIGS. 2A and 2B. In particular, FIG. 2A is a plot of overall survival (OS) for patients in the gefitinib (monotherapy) arm of the Study. FIG. 2B is a plot of the progression free survival (PFS) for patients in the monotherapy arm of the Study. FIGS. 2A and 2B illustrate that the VeriStrat signature (“good”/“poor”) is prognostic for OS and PFS in the gefitinib arm, i.e., there is a clear difference in both PFS and OS outcomes for both PFS and OS between the VeriStrat good and VeriStrat poor patients, with VeriStrat good patients having greater PFS and OS as compared to the VeriStrat poor patients. Note that in FIG. 2A those patients testing VeriStrat poor have much worse OS and PFS as compared to those patients whose serum tested as VeriStrat good. FIGS. 2A and 2B are consistent with our earlier studies described in U.S. Pat. No. 7,736,905.

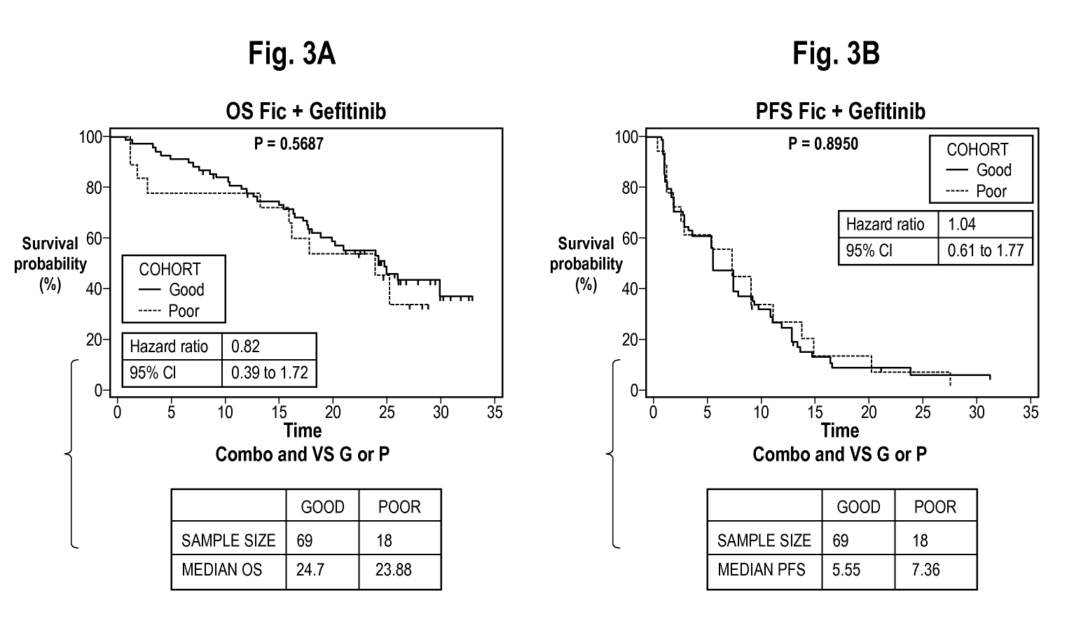

FIG. 3A is a Kaplan-Meier plot of OS for patients in the gefitinib+ficlatuzumab arm of the Study. FIG. 3B is a plot of the PFS for patients in the gefitinib+ficlatuzumab arm of the Study. FIGS. 3A and 3B illustrate that the VeriStrat signature (“good”/“poor”) was not prognostic for OS and PFS in the gefitinib+ficlatuzumab arm, i.e., there is no difference is outcomes between the curves for good and poor patients. That is, those patients who were treated with the combination therapy and which tested VeriStrat poor prior to treatment had very similar OS and PFS as those patients who tested VeriStrat good prior to treatment and were also treated with the combination therapy.

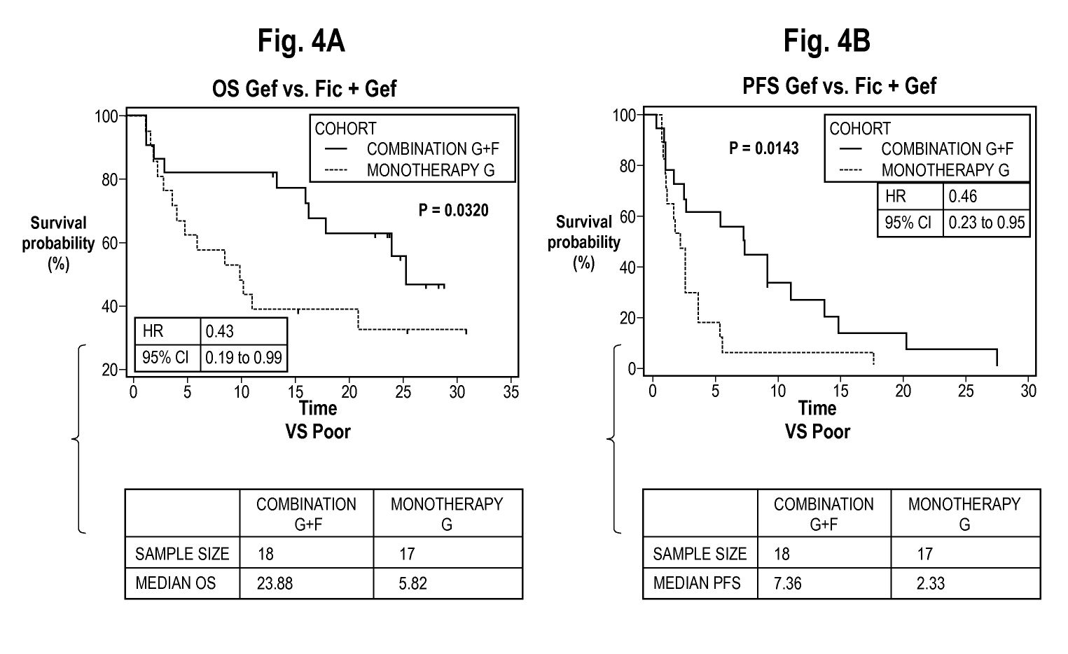

The Kaplan-Meier plots of FIGS. 4A and 4B are particularly significant. FIG. 4A is a plot of OS for patients in the gefitinib+ficlatuzumab combination treatment arm compared to the gefitinib monotherapy arm, for those patients with VeriStrat poor status. FIG. 4B is a plot of PFS for patients in the gefitinib+ficlatuzumab combination arm compared to the gefitinib monotherapy arm, for those patients with VeriStrat poor status. FIGS. 4A and 4B illustrate that the patients testing VeriStrat poor in advance of treatment were likely to benefit from the addition of ficlatuzumab to gefitinib relative to gefitinib monotherapy. Comparing FIGS. 4A and 4B to FIGS. 2A-2B and 3A-3B, it is evident that VeriStrat poor signature in NSCLC patients, obtained from serum samples pre-treatment, indicates that such patients are more likely to benefit from the addition of a monoclonal antibody drug targeting HGF, such as ficlatuzumab, to an EGFR-I, such as gefitinib in treatment of the cancer relative to EGFR-I monotherapy. Note that in the combination therapy arm, the median survival of the poor patients was 23.88 months (95% CI 13.26—not evaluable), whereas in the monotherapy arm the mean overall survival was only 5.82 months (95% CI 2.17-10.95). The median progression free survival of the VeriStrat poor patients in the combination therapy arm was 7.36 months (95% CI 1.77-11.11), whereas in the monotherapy arm the median progression free survival of the VeriStrat poor patients was only 2.33 months (95% CI 1.08-3.68).

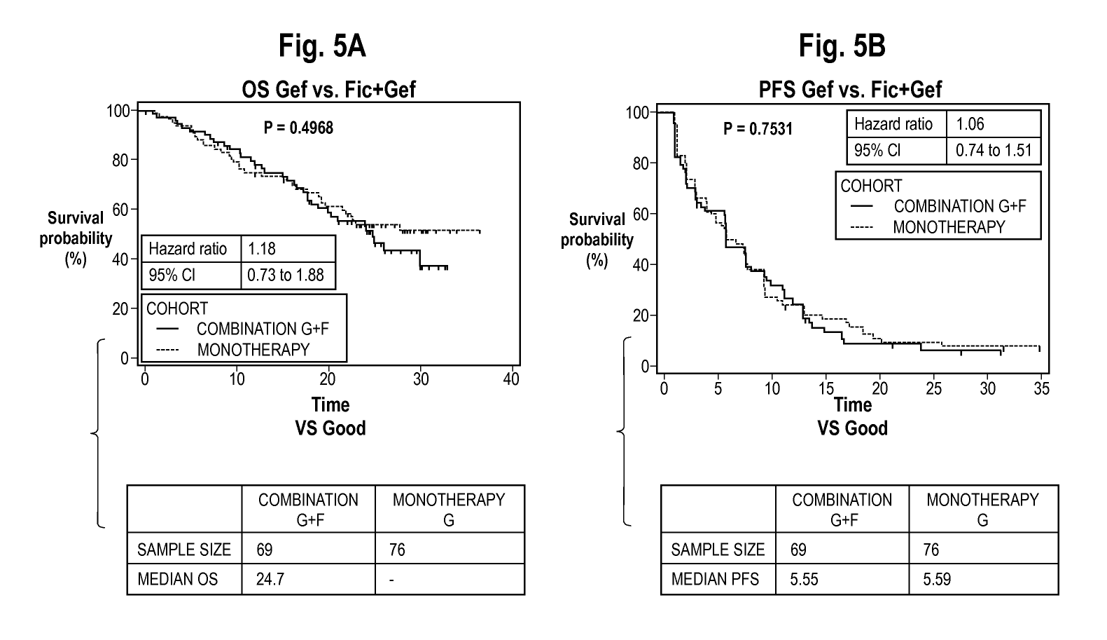

FIG. 5A is a plot of OS for patients in the gefitinib+ficlatuzumab arm as compared to the gefitinib monotherapy arm, for those patients with VeriStrat “good” status. FIG. 5B is a plot of PFS for patients in the gefitinib+ficlatuzumab arm as compared to the gefitinib monotherapy arm. FIGS. 5A and 5B illustrate that the patients testing VeriStrat good in advance of treatment appear to derive no increased benefit from addition of ficlatuzumab to gefitinib.

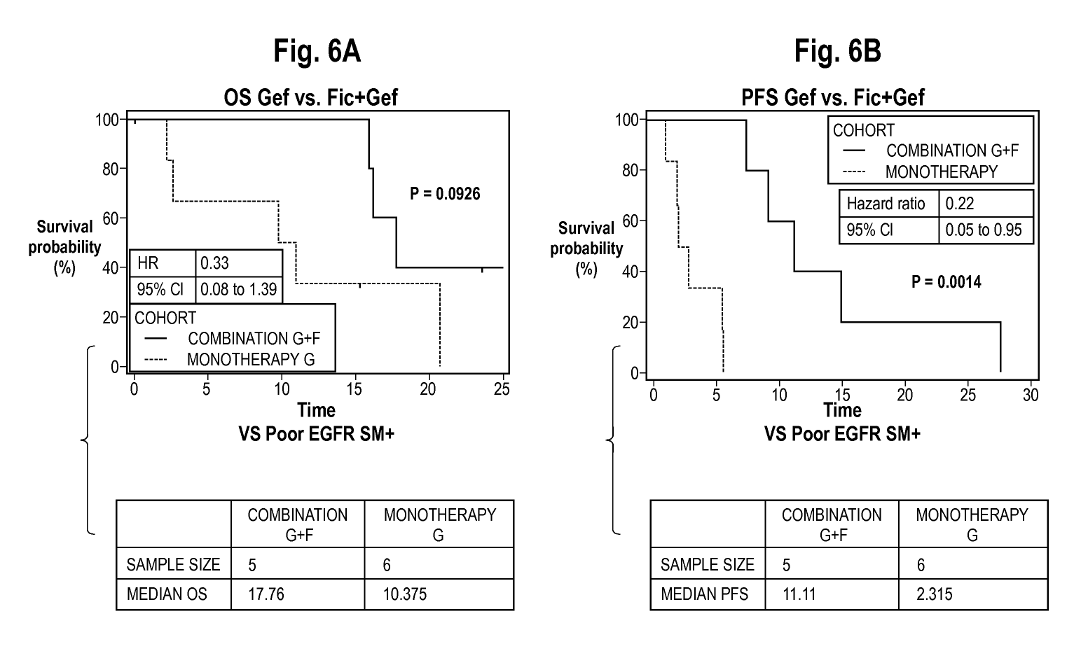

FIG. 6A is a plot of OS for patients in the gefitinib+ficlatuzumab arm as compared to the gefitinib monotherapy arm, for those patients with (i) VeriStrat poor status pre-treatment and (ii) having EGFR sensitizing mutations (EGFR SM+) such as exon 19 deletion or substitutions at L858R, G719X or L861Q). FIG. 6B is a plot of the progression free survival (PFS) for patients in the gefitinib+ficlatuzumab arm compared to the gefitinib monotherapy arm, for this same group of patients. Note, that the number of patients in the groups are small, and consequently, the results should be interpreted with caution. FIGS. 6A and 6B illustrate that patients testing VeriStrat poor pre-treatment, and have EGFR SM+ status, were likely to benefit in PFS (p=0.014) from the addition of ficlatuzumab to gefitinib compared to gefitinib monotherapy, while in OS the difference did not reach statistical significance (p=0.0926).

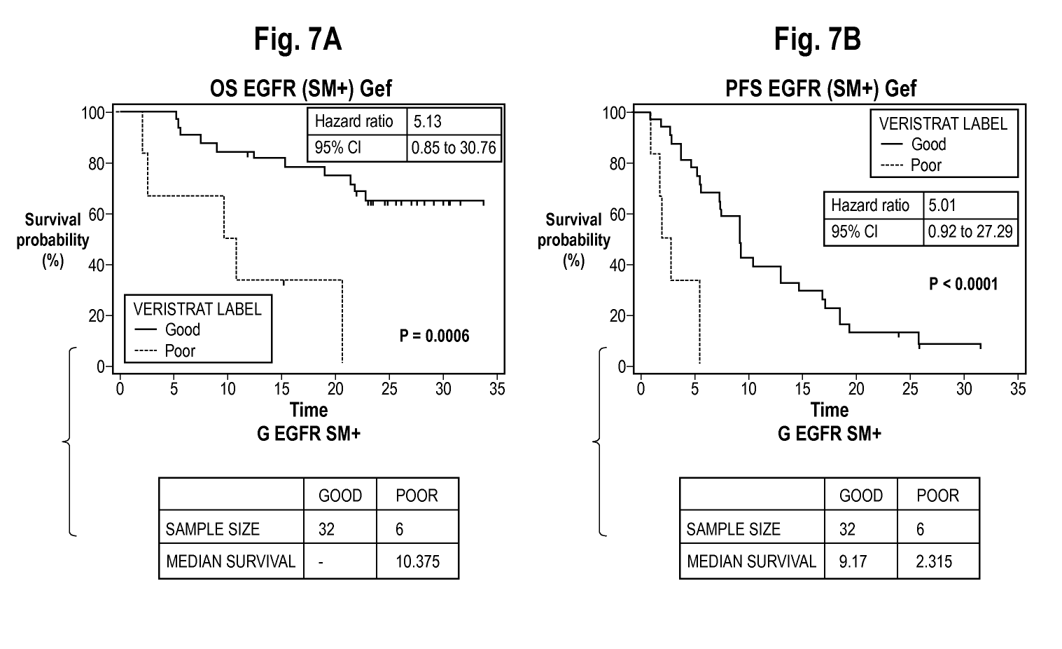

FIG. 7A is a plot of OS for patients in the gefitinib arm for those patients with VeriStrat poor and VeriStrat good status, and having EGFR SM+ patients. FIG. 7B is a plot of the PFS for patients in the gefitinib arm for those patients with VeriStrat poor and VeriStrat good status, and having EGFR SM+ status. These plots show that, despite having EGFR SM+ status, those patients also testing VeriStrat poor did significantly worse than those patients testing VeriStrat good.

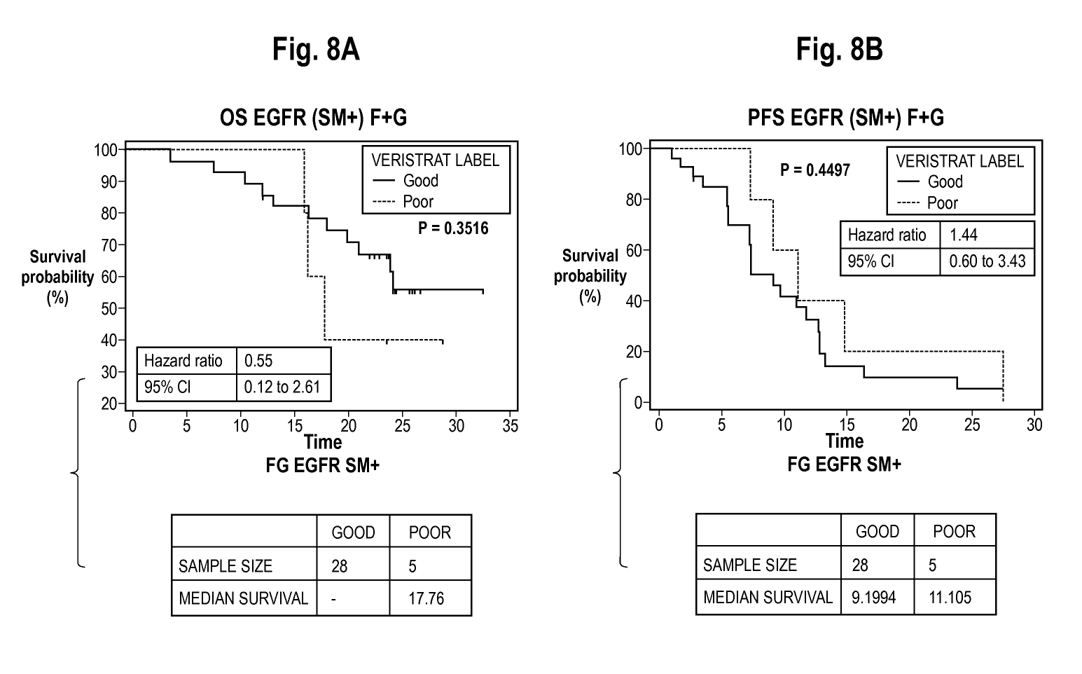

FIG. 8A is a plot of OS for patients in the gefitinib+ficlatuzumab combination arm for those patients with VeriStrat poor and VeriStrat good status, and having EGFR SM+ status. FIG. 8B is a plot of the PFS for patients in the gefitinib+ficlatuzumab combination arm for those patients with VeriStrat poor and VeriStrat good status, and having EGFR SM+ status. There were no significant difference between VeriStrat good and VeriStrat poor patients in OS (p=0.3516) or PFS (p=0.4497) in the combination arm. By comparing FIG. 8B with FIG. 7B, we also note that the median PFS for the poor patients in the combination arm is 11.1 months (95% CI 7.36-27.56), compared to 2.3 months (95% CI 0.95-5.52) in the monotherapy arm.

The interpretation of data from small sample sets like the one above is always confounded by sample set bias. We are therefore considering the presented results only as an indication supporting the benefit of the addition of ficlatuzumab to gefitinib over gefitinib alone in EGFR mutation positive patients, and not as the sole evidence for the claims in this disclosure. For example, variations in sample collection times could lead to small percentages of changes in VeriStrat labels, which could easily affect the significance of the presented data. Also subsets of the data may modify such fragile statistical considerations. As an example we provide some results for a subset of patients who had samples available collected prior to the ones used in the above analysis.

The pre-treatment serum samples used in the foregoing analysis were largely derived from patient blood samples originally drawn immediately prior to drug dosing (hereafter called “C1D1” samples) in order to define baselines for drug pharmacokinetics and pharmacodynamics. Subsequently, a set of blood samples was analyzed, these being derived from blood draws taken from 1-12 days (median 4.4 days) prior to drug dosing (hereafter called “SCR” samples) for purposes of establishing patient eligibility for study with respect to blood chemistries. A total of 165 patients, which is a subset of the data analyzed from the whole study, provided appropriately consented samples from both the SCR and C1D1 draws allowing comparison of VeriStrat status between the sample sets for the subset of patients where SCR samples were available

Although the concordance between these two sample sets was high at 90%, a small 10% discordance changed the composition of VeriStrat poor status among patients in the treatment groups. The C1D1 set originally analyzed contained 35 apparently VeriStrat poor patients in the ITT population (18 who received gefitinib+ficlatuzumab; 17 who received gefitinib alone), and 11 in the EGFR SM+ population (5 who received ficlatuzumab+gefitinib and 6 who received gefitinib alone). The SCR contained 31 VeriStrat poor patients in the ITT population (13 who received gefitinib+ficlatuzumab and 18 who received gefitinib alone), and 10 patients in the EGFR SM+ population (2 who received gefitinib+ficlatuzumab and 8 who received gefitinib alone). Especially this last observation renders a statistical analysis of the SCR set meaningless.

Analysis of the SCR data yielded statistically indistinguishable results to the C1D1 data insofar as SCR hazard ratios and medians were within the 95% confidence interval of those observed in the C1D1 data. In the SCR ITT population, median PFS for VeriStrat poor patients on gefitinib+ficlatuzumab was 5.5 months vs. 2.7 months for VeriStrat poor patients on gefitinib alone (H.R. 0.68; p=0.29). For the SCR EGFR SM+ population, median PFS for VeriStrat poor patients on gefitinib+ficlatuzumab was 7.4 months vs. 4.1 months for VeriStrat poor patients on gefitinib alone (H.R. 0.8; p=0.33). Insofar as both estimates of the relative benefit of gefitinib+ficlatuzumab vs. gefitinib alone in VeriStrat poor patients are based on very small sample sizes, a more accurate estimate of the magnitude of clinical benefit awaits a larger clinical study.

Testing Method

The methods of this disclosure for identifying a NSCLC patient who is likely to obtain benefit from administration of combination therapy in the form of EGFR-I and a monoclonal antibody drug targeting HGF, as compared to EGFR-I monotherapy, involves obtaining a serum or plasma sample from the NSCLC lung cancer patient and processing it in accordance with the test described in this section of this document. The result of the test is a class label that is assigned to the specimen, and which indicates whether the patient is likely to benefit from the combination therapy. That is, if the class label is “poor” or the equivalent, the patient is predicted as being likely to benefit, whereas if the label is “good” or the equivalent, the patient is predicted to be unlikely to benefit from addition of an HGF-targeting monoclonal antibody relative EGFR-I treatment alone, i.e., the good patients are predicted to have similar outcomes from either the EGFR-I monotherapy or the combination therapy.

The test is illustrated in flow chart form in FIG. 9 as a process step 100. At step 102, a serum or plasma sample is obtained from the patient. In one embodiment, the serum samples are separated into three aliquots and the mass spectroscopy and subsequent steps 104, 106 (including sub-steps 108, 110 and 112), 114, 116 and 118 are performed independently on each of the aliquots. The number of aliquots can vary, for example there may be 4, 5 or 10 aliquots, and each aliquot is subject to the subsequent processing steps.

At step 104, the sample (aliquot) is subject to mass spectroscopy. A preferred method of mass spectroscopy is matrix assisted laser desorption ionization (MALDI) time of flight (TOF) mass spectroscopy. Mass spectroscopy produces data points that represent intensity values at a multitude of mass/charge (m/z) values, as is conventional in the art. In one example embodiment, the samples are thawed and centrifuged at 1500 rpm for five minutes at four degrees Celsius. Further, the serum samples may be diluted 1:10, or 1:5, in MilliQ water. Diluted samples may be spotted in randomly allocated positions on a MALDI plate in triplicate (i.e., on three different MALDI targets). After 0.75 ul of diluted serum is spotted on a MALDI plate, 0.75 ul of 35 mg/ml sinapinic acid (in 50% acetonitrile and 0.1% trifluoroacetic acid (TFA)) may be added and mixed by pipetting up and down five times. Plates may be allowed to dry at room temperature. It should be understood that other techniques and procedures may be utilized for preparing and processing serum in accordance with the principles of the present invention.

Mass spectra may be acquired for positive ions in linear mode using a Voyager DE-PRO or DE-STR MALDI TOF mass spectrometer with automated or manual collection of the spectra. (Other mass spectrometers may also be used). Two thousand shot filtered spectra are acquired from each serum specimen. Spectra are externally calibrated using a mixture of protein standards (Insulin (bovine), thioredoxin (E. coli), and Apomyglobin (equine)).

At step 106, the spectra obtained in step 104 are subject to pre-defined pre-processing steps. The pre-processing steps 106 are implemented in a general purpose computer using software instructions that operate on the mass spectral data obtained in step 104. The pre-processing steps 106 include background subtraction (step 108), normalization (step 110) and alignment (step 112). The step of background subtraction preferably involves generating a robust, asymmetrical estimate of background in the spectrum and subtracts the background from the spectrum. Step 108 uses the background subtraction techniques described in U.S. Pat. No. 7,736,905, which is incorporated by reference herein. The normalization step 110 involves a normalization of the background subtracted spectrum. The normalization can take the form of a partial ion current normalization, or a total ion current normalization, as described in U.S. Pat. No. 7,736,905. Step 112 aligns the normalized, background subtracted spectrum to a predefined mass scale, as described in U.S. Pat. No. 7,736,905, which can be obtained from investigation of the training set used by the classifier.

Once the pre-processing steps 106 are performed, the process 100 proceeds to step 114 of obtaining integrated intensity values of selected features in the spectrum over predefined m/z ranges. The normalized and background subtracted amplitudes may be integrated over these m/z ranges and assign this integrated value (i.e., the area under the curve within the range of the feature) to a feature. This step is also disclosed in further detail in U.S. Pat. No. 7,736,905.

At step 114, as described in U.S. Pat. No. 7,736,905, the integrated values of features in the spectrum are obtained from the following m/z ranges:

5732 to 5795
5811 to 5875
6398 to 6469
11376 to 11515
11459 to 11599
11614 to 11756
11687 to 11831
11830 to 11976
12375 to 12529
23183 to 23525
23279 to 23622 and
65902 to 67502.

In a preferred embodiment, values are obtained at eight m/z ranges which encompass the peaks listed in Table 3 below. The significance, and methods of discovery of these ranges, is explained in the U.S. Pat. No. 7,736,905.

At step 116, the values obtained at step 114 are supplied to a classifier, which in the illustrated embodiment is a K-nearest neighbor (KNN) classifier. The classifier makes use of a reference set of class labeled spectra from a multitude of other patients, which in the preferred embodiment are NSCLC cancer patients. Digital data representing the reference set should be previously obtained and stored in memory accessible to the general purpose computer executing the classification step 116, e.g., stored in a hard disk memory, database or cloud accessible to the computer. The classification algorithm essentially consists of a majority vote algorithm that compares the integrated intensity values obtained in step 114 to the intensity values of K nearest neighbors in a multi-dimensional feature space formed by the reference set using a Euclidean distance. The value of K in the KNN algorithm was chosen to be 7 but similar tests be obtained for K=3, 5, or other suitable values. The application of the KNN classification algorithm to the values at 114 and the reference set is explained in U.S. Pat. No. 7,736,905. Other classifiers can be used, including a probabilistic KNN classifier, margin-based classifier, or other type classifier and might lead to different but similarly performing tests. K-Nearest neighbor classification algorithms are well known in the art and the particular details are not necessary for the present discussion. The reference set was constructed by combining specific sample sets from our previous NSCLC work and assigning class labels as follows: A class label “poor” was assigned to those patients who had early progression after treatment with an EGFR-I, and a class label “good” was assigned to those that had stable disease longer than 6 months after treatment with an EGFR-I. The reason for using the NSCLC reference set we also used in the VeriStrat test of U.S. Pat. No. 7,736,905 for the present study is that it has been well characterized and subject to extensive validation. However, it is theoretically possible to construct a training set and to validate it from test spectra obtained from a multitude of other types of solid epithelial cancer patients, for example patients having CRC, SCCHN, resulting in different but similarly performing tests. In these alternative embodiments, the training set labels would similarly be “good” or “poor”, the “good” and “poor” class labels assigned as explained previously in this paragraph.

At step 118, the classifier produces a label for the spectrum, either “Good”, “Poor” or “Indeterminate.” As mentioned above, steps 104-118 are performed separately on the three separate aliquots from a given patient sample (or whatever number of aliquots are used). At step 120, a check is made to determine whether all the aliquots produce the same class label. If not, an Indeterminate result is returned as indicated at step 122. If all aliquots produce the same label, the label is reported as indicated at step 124.

As described in this document, new and unexpected uses of the class label reported at step 124 are disclosed. In particular, those NSCLC patients whose serum or plasma samples are labeled “Poor” in accordance with the VeriStrat test are likely to benefit from combination treatment in the form of addition of a monoclonal antibody drug targeting HGF (e.g. ficlatazumab or the equivalent), in addition to an EGFR-I such as gefitinib as compared to EGFR-I monotherapy.

It will be understood that steps 106, 114, 116 and 118 are typically performed in a programmed general purpose computer using software coding the pre-processing step 106, the obtaining of spectral values in step 114, the application of the K-NN classification algorithm in step 116 and the generation of the class label in step 118. The training set of class labeled spectra used in step 116 is stored in memory in the computer or in a memory accessible to the computer, e.g., in associated database, cloud storage, or loaded on portable computer readable medium.

The method and programmed computer may be advantageously implemented at a laboratory test processing center as described in U.S. Pat. No. 7,736,905 and conducting testing of serum or plasma samples for NSCLC patients as a fee for service.

TABLE 3
Peaks used in VeriStrat.

| Peak number | m/z |
|---|---|
| 1 | 5843 |
| 2 | 11445 |
| 3 | 11529 |
| 4 | 11685 |
| 5 | 11759 |
| 6 | 11903 |
| 7 | 12452 |
| 8 | 12579 |

Other Mass Spectrometry and Classification Methods

While the disclosed embodiments have been described in conjunction with the m/z features referenced in our prior U.S. Pat. No. 7,736,905, it will be understood that it is possible to perform classification on the basis of distinguishing m/z features obtained from mass spectra using the so-called deep-MALDI methods. In these methods, mass spectra from the sample are obtained from at least 20,000 laser shots in MALDI-TOF mass spectrometry. This method is described in the US patent application of H. Röder et al., publication no. 2013/0320203, the content of which is incorporated by reference herein, and Duncan, et al., Extending the Information Content of the MALDI Analysis of Biological Fluids (Deep MALDI) presented at 61st ASMS Conference on Mass Spectrometry and Allied Topics, Minneapolis, USA June 2013. In this method, as explained in the '203 patent application publication, many more spectral features are revealed in serum or plasma as compared to the typical 500 to 2000 shot spectra obtained in typical “dilute and shoot” MALDI-TOF mass spectrometry.

Furthermore, a classifier can be generated from spectra using the classifier generation methods of US application of H. Röder et al., Ser. No. 14/486,442 filed Sep. 15, 2014 entitled “Classification method using combination of mini-classifiers with dropout and uses thereof,” which is incorporated by reference herein. The methods of the '442 application create classifiers that are a regularized combination of a filtered set of mini-classifiers. The classifiers can be created from mass spectral feature obtained with either “dilute and shoot” or “deep-MALDI” methods.

Treatment Methods

It will be appreciated from this disclosure that we have also described a method of treating a NSCLC patient. The treatment is in the form of administrating to the patient a combination of an EGFR-I, e.g., gefitinib, and a monoclonal antibody drug targeting HGF, e.g., a monoclonal antibody targeting HGF such as ficlatazumab. The patient is selected for such administration in advance by conducting a test in the form of the following steps of:

(a) providing a serum or plasma sample from the NSCLC patient to a mass spectrometer and conducting mass spectrometry on the serum or plasma sample and thereby generating a mass spectrum for the serum or plasma sample; (See FIG. 9, steps 102, 104)

(b) conducting pre-defined pre-processing steps on the mass spectrum obtained in step (a) with the aid of a programmed computer, such as for example background subtraction, normalization and alignment; (FIG. 9, step 106)

(c) obtaining integrated intensity values of selected features in said mass spectrum at one or more predefined m/z ranges after the pre-processing steps on the mass spectrum recited in step (c) have been performed; (FIG. 9 step 114) and

(d) executing in the programmed computer a classification algorithm operating on both the integrated intensity values obtained in step (c) and a reference set comprising data in the form of class-labeled mass spectra obtained from a multitude of cancer patients stored in a computer readable medium accessible by the programmed computer, (FIG. 9 step 116). The class-labels in the reference set are of the form GOOD (or the equivalent) and POOR (or the equivalent) as defined previously. The method includes the sub-step of generating a class label for the serum or plasma sample (FIG. 9 step 118). As explained above in conjunction with FIGS. 2A-2B, 4A-4B, 5A-5B, 6A-6B, if the class label generated in step (d) is POOR or the equivalent for the serum or plasma sample, the patient is identified as being likely to benefit from the combination treatment more than from EGFR-I monotherapy.

In one embodiment, the EGFR-I is in the form of gefitinib or similar small molecule EGFR-I drugs e.g., erlotinib, and so-called second generation EGFR-Is such as afatinib. In one specific embodiment, the monoclonal antibody drug binds to HGF and may be ficlatuzumab or the equivalent.

In one specific embodiment, the reference set used for classification is in the form of data representing class-labeled mass spectra obtained from a multitude of NSCLC patients. The classification algorithm in one embodiment is in the form of a k-nearest neighbor classification algorithm. In one specific embodiment, the predefined m/z ranges used for classification of the sample mass spectrum include one or more of the m/z peaks listed in TABLE 3, for example the m/z ranges encompassing all 8 peaks.

The skilled clinician will be able to determine the appropriate dosage amount and number of doses of agents to be administered to a subject, dependent upon both the age and weight of the subject, the underlying condition, and the response of an individual subject to the treatment. In addition, the clinician will be able to determine the appropriate timing and routes for delivery of the agent in a manner effective to treat the subject. Dosing may be done consistent with FDA-approved labeling or in accordance with clinical experience. An exemplary dose for gefitinib is a 250 mg tablet as a daily dose. Exemplary doses for erlotinib are a 25 mg, 100 mg or 150 mg tablet as a daily dose. An exemplary dosage regimen for cetuximab is 400 mg/m2 as an initial dose as a 120 minute intravenous infusion followed by 250 mg/m2 weekly, infused over 60 minutes.

Exemplary dosage regimens for ficlatuzumab are 2 mg/kg every two weeks, 10 mg/kg, every 2 weeks, and 20 mg/kg, every 2 weeks, which is administered parenterally, e.g., by intravenous infusion.

In another aspect, a method of treating a subject with Non-Small Cell Lung Cancer (NSCLC) who are likely to benefit more from the combination treatment than from EGFR-I monotherapy is disclosed. The method comprises the steps of:

(1) determining whether said subject with NSCLC is a member of a class of cancer patients likely to benefit from a treatment for NSCLC in the form of administration of a combination of an EGFR-I and a monoclonal antibody drug targeting hepatocyte growth factor (HGF) as compared to treatment with EGFR-I monotherapy using the following steps (a)-(e):

(a) storing in a computer readable medium a reference set comprising non-transient data in the form of class-labeled mass spectral data obtained from a multitude of cancer patients, the class-labels of the form Good or the equivalent and Poor of the equivalent, the meaning of Good and Poor class labels as explained above,

(b) providing a serum or plasma sample from the NSCLC patient to a mass spectrometer and conducting mass spectrometry on the serum or plasma sample and thereby generating a mass spectrum for the serum or plasma sample;

(c) conducting pre-defined pre-processing steps on the mass spectrum obtained in step b) with the aid of a programmed computer;

(d) obtaining integrated intensity values of selected features in said mass spectrum over predefined m/z ranges after the pre-processing steps on the mass spectrum recited in step c) have been performed; and

(e) executing in the programmed computer a classification algorithm operating on both the integrated intensity values obtained in step (d) and the reference set stored in step (a) and responsively generating a class label for the serum or plasma sample,

wherein if the class label generated in step (e) is POOR or the equivalent for the blood based sample the patient is identified as being a member of the class as likely to benefit from the combination treatment as compared to monotherapy; and

(2) if the subject is identified as being a member of the class with the class label of POOR or the equivalent, treating the subject with a combination of an EGFR-I and the monoclonal antibody drug targeting HGF.

In still another aspect, a method of treating a subject with Non-Small Cell Lung Cancer (NSCLC) is disclosed, the method comprising the step of administering a combination of an effective amount of the EGFR-I and the monoclonal antibody drug targeting HGF to a subject predicted by mass spectrometry of a serum or plasma sample to be a member of a class of patients likely to benefit from epidermal growth factor receptor inhibitor (EGFR-I) in combination with a monoclonal antibody drug targeting hepatocyte growth factor (HGF), as compared to EGFR-I monotherapy alone.

In another aspect, a method of treating a subject with Non-Small Cell Lung Cancer (NSCLC) is disclosed, the method comprising the steps of: administering to a subject identified by performing steps (a)-(e) that is likely to benefit from a combination therapy comprising an epidermal growth factor receptor inhibitor (EGFR-I) and a monoclonal antibody drug targeting hepatocyte growth factor (HGF) as compared to monotherapy a combination of an effective amount of the EGFR-I and the monoclonal antibody drug targeting HGF; wherein steps (a)-e) comprise the steps of:

(a) storing in a computer readable medium a reference set comprising non-transient data in the form of class-labeled mass spectral data obtained from a multitude of cancer patients, the class-labels indicating whether the patients associated with the mass spectral data did or did not belong to class label GOOD or the equivalent, or class label POOR or the equivalent, the meaning of the Good and Poor class labels as explained above,

(b) providing a serum or plasma sample from the NSCLC patient to a mass spectrometer and conducting mass spectrometry on the serum or plasma sample and thereby generating a mass spectrum for the serum or plasma sample;

(c) conducting pre-defined pre-processing steps on the mass spectrum obtained in step b) with the aid of a programmed computer;

(d) obtaining integrated intensity values of selected features in said mass spectrum over predefined m/z ranges after the pre-processing steps on the mass spectrum recited in step c) have been performed; and

(e) executing in the programmed computer a classification algorithm operating on both the integrated intensity values obtained in step (d) and intensity values of the reference set stored in step (a) and responsively generating a class label for the serum or plasma sample,

wherein if the class label generated in step e) is POOR for the blood based sample the patient is identified as being likely to benefit from the combination treatment as compared to monotherapy.

In the method of treatment, in one embodiment subject is treated with the combination of an EGFR-I selected from the group consisting of gefitinib, erlotinib and cetuximab and a monoclonal antibody drug that binds to HGF. In one embodiment, the monoclonal antibody is ficlatuzumab or the equivalent, e.g., generic version thereof. The “equivalent” here is used to encompass, for example, a generic version of ficlatuzumab, or another Mab that binds to HGF but has a different physical structure or composition but otherwise performs the substantially the same function to bind to the MET receptor substantially the same way to achieve the same overall result of inhibiting MET.

The appended claims are further descriptions of the disclosed inventions.

What is claimed is:

1. A method for predicting whether a NSCLC patient is a member of a class of cancer patients likely to benefit from a treatment for NSCLC in the form of administration of a combination of an epidermal growth factor receptor inhibitor (EGFR-I) and a monoclonal antibody drug targeting hepatocyte growth factor (HGF) as compared to EGFR-I monotherapy comprising the steps of:
(a) storing in a computer readable medium a reference set comprising non-transient data in the form of class-labeled mass spectral data obtained from a multitude of cancer patients, the class-labels of the form GOOD or the equivalent indicating the patient had stable disease six months after initiating treatment of the cancer with an EGFR-I and POOR or the equivalent indicating the patients had early progression of disease after initiating treatment of the cancer with an EGFR-I;
(b) providing a serum or plasma sample from the NSCLC patient to a mass spectrometer and conducting mass spectrometry on the serum or plasma sample and thereby generating a mass spectrum for the serum or plasma sample;
(c) conducting pre-defined pre-processing steps on the mass spectrum obtained in step b) with the aid of a programmed computer;
(d) obtaining integrated intensity values of selected features in said mass spectrum at one or more predefined m/z ranges after the pre-processing steps on the mass spectrum recited in step c) have been performed; and
(e) executing in the programmed computer a classification algorithm operating on both the integrated intensity values obtained in step (d) and the reference set stored in step (a) and responsively generating a class label for the serum or plasma sample,
wherein if the class label generated in step e) is POOR or the equivalent for the serum or plasma sample the patient is identified as being likely to benefit from the combination treatment.

2. The method of claim 1, wherein the EGFR-I comprises gefitinib or similar small molecule drugs targeting EGFR.

3. The method of claim 1, wherein the monoclonal antibody drug targeting HGF comprises a monoclonal antibody designed to bind to HGF.

4. The method of claim 3, wherein the drug comprises ficlatuzumab or the equivalent.

5. The method of claim 1, wherein the reference set comprises class-labeled mass spectra obtained from a multitude of NSCLC patients.

6. The method of claim 1, wherein the classification algorithm comprises a k-nearest neighbor classification algorithm.

7. The method of claim 1, wherein the predefined m/z ranges encompass one or more m/z peaks listed in TABLE 3.

8. The method of claim 1, wherein the classification algorithm uses a regularized combination of a filtered set of mini-classifiers.

9. A method of treating a subject with Non-Small Cell Lung Cancer (NSCLC) who is not likely to benefit from monotherapy treatment with an epidermal growth factor receptor inhibitor (EGFR-I), the method comprising:
(1) determining whether said subject with NSCLC is a member of a class of cancer patients likely to benefit from a treatment for NSCLC in the form of administration of a combination of an EGFR-I and a monoclonal antibody drug targeting hepatocyte growth factor (HGF) using the following steps (a)-(e):
(a) storing in a computer readable medium a reference set comprising non-transient data in the form of class-labeled mass spectral data obtained from a multitude of cancer patients, the class-labels of the form GOOD or the equivalent indicating the patient had stable disease six months after initiating treatment of the cancer with an EGFR-I and POOR or the equivalent indicating the patients had early progression of disease after initiating treatment of the cancer with an EGFR-I;
(b) providing a serum or plasma sample from the NSCLC patient to a mass spectrometer and conducting mass spectrometry on the serum or plasma sample and thereby generating a mass spectrum for the serum or plasma sample;
(c) conducting pre-defined pre-processing steps on the mass spectrum obtained in step (b) with the aid of a programmed computer;
(d) obtaining integrated intensity values of selected features in said mass spectrum at one or more predefined m/z ranges after the pre-processing steps on the mass spectrum recited in step (c) have been performed; and
(e) executing in the programmed computer a classification algorithm operating on both the integrated intensity values obtained in step (d) and the reference set stored in step (a) and responsively generating a class label for the serum or plasma sample,
wherein if the class label generated in step (e) is POOR or the equivalent for the blood based sample the patient is identified as being likely to benefit from the combination treatment; and
(2) if the subject is identified as being a member of the class with the class label of POOR or the equivalent, treating the subject with a combination of an EGFR-I and the monoclonal antibody drug targeting HGF.

10. A method of treating a subject with Non-Small Cell Lung Cancer (NSCLC), the method comprising: administering to a subject, predicted by mass spectrometry of a blood-based sample to be a member of a class of patients unlikely to benefit from epidermal growth factor receptor inhibitor (EGFR-I) monotherapy, treatment in the form of a combination of an EGFR-I and a monoclonal antibody drug targeting hepatocyte growth factor (HGF).

11. A method of treating a subject with Non-Small Cell Lung Cancer (NSCLC), the method comprising: administering to a subject identified by performing steps (a)-(e) that is likely to benefit from a combination therapy comprising an epidermal growth factor receptor inhibitor (EGFR-I) and a monoclonal antibody drug targeting hepatocyte growth factor (HGF) a combination of an effective amount of the EGFR-I and the monoclonal antibody drug targeting HGF; wherein steps (a)-e) comprise the steps of:
(a) storing in a computer readable medium a reference set comprising non-transient data in the form of class-labeled mass spectral data obtained from a multitude of cancer patients, the class-labels of the form GOOD or the equivalent indicating the patient had stable disease six months after initiating treatment of the cancer with an EGFR-I and POOR or the equivalent indicating the patients had early progression of disease after initiating treatment of the cancer with an EGFR-I;
(b) providing a blood-based sample from the NSCLC patient to a mass spectrometer and conducting mass spectrometry on the blood-based sample and thereby generating a mass spectrum for the blood-based sample;
(c) conducting pre-defined pre-processing steps on the mass spectrum obtained in step b) with the aid of a programmed computer;
(d) obtaining integrated intensity values of selected features in said mass spectrum at one or more predefined m/z ranges after the pre-processing steps on the mass spectrum recited in step c) have been performed; and
(e) executing in the programmed computer a classification algorithm operating on both the integrated intensity values obtained in step (d) and the reference set stored in step (a) and responsively generating a class label for the blood-based sample,
wherein if the class label generated in step (e) is POOR or the equivalent for the blood based sample the patient is identified as being likely to benefit from the combination treatment.

12. The method of claim 9, wherein the subject is treated with the combination of an EGFR-I selected from the group consisting of gefitinib, erlotinib and cetuximab and a monoclonal antibody drug that binds to HGF.

13. The method of claim 10, wherein the subject is treated with the combination of an EGFR-I selected from the group consisting of gefitinib, erlotinib and cetuximab and a monoclonal antibody drug that binds to HGF.

14. The method of claim 11, wherein the subject is treated with the combination of an EGFR-I selected from the group consisting of gefitinib, erlotinib and cetuximab and a monoclonal antibody drug that binds to HGF.

15. The method of claim 12, wherein the monoclonal antibody is ficlatuzumab or the equivalent.

16. The method of claim 13, wherein the monoclonal antibody is ficlatuzumab or the equivalent.

17. The method of claim 14, wherein the monoclonal antibody is ficlatuzumab or the equivalent.
